(19) United States
(12) Patent Application Publication
(10) Pub. No.: US 2025/0287953 A1
(43) Pub. Date: Sep. 18, 2025

(54) ANTIMICROBIAL WATER-DISPERSIVE COMPOSITION INCLUDING COPPER CITRATE AS ACTIVE INGREDIENT

(71) Applicant: CHEMICEUTICAL INC., Anyang-si, Gyeonggi-do (KR)

(72) Inventor: In Soo PARK, Anyang-si, Gyeonggi-do (KR)

(73) Assignee: CHEMICEUTICAL INC., Anyang-si, Gyeonggi-do (KR)

(21) Appl. No.: 18/283,366

(22) PCT Filed: Mar. 15, 2022
(86) PCT No.: PCT/KR2022/003593

§ 371 Date: Jul. 5, 2024

(30) Foreign Application Priority Data
Mar. 23, 2021 (KR) 10-2021-0037106

(51) Int. Cl. A01N 59/20; A01P 1/00; A61K 8/362; A61Q 17/00; C09D 5/14

(52) U.S. Cl. CPC A01N 59/20; A01P 1/00; A61K 8/362; A61Q 17/005; C09D 5/14

(57) ABSTRACT

An antimicrobial water-dispersive composition includes copper citrate as an active ingredient. The antimicrobial water-dispersive composition has excellent water dispersion stability and exhibits a wide antibacterial spectrum against bacteria, fungi and viruses when added to personal care products and paints. Thus, personal care products and paints containing the antimicrobial water-dispersive composition are also disclosed. The copper citrate is a copper citrate hydride or copper citrate anhydride.

### CROSS REFERENCE TO RELATED APPLICATIONS

This application is a National Stage of International Application No. PCT/KR2022/003593 filed Mar. 15, 2022, claiming priority based on Korean Patent Application No. 10-2021-0037106 dated Mar. 23, 2021, and the entire contents of which are incorporated as part of this specification.

### TECHNICAL FIELD

The present invention relates to an antimicrobial water-dispersive composition including copper citrate as an active ingredient. More specifically, it relates to an antimicrobial water-dispersive composition including copper citrate as an active ingredient that has excellent water dispersion stability and exhibits a wide antibacterial spectrum against bacteria, fungi and viruses when added to personal care products and paints.

### BACKGROUND ART

Following the recent global pandemic of coronavirus infection (COVID-19), each paint manufacturer in the world is competitively releasing architectural paints that can kill the corona virus. The active ingredients of these paints are gold, silver, copper, etc. in their elemental state, and the active substances that realize actual antiviral performance are known as ionic components of these metals, which are produced in extremely small amounts when these metal elements come into contact with moisture in the air.

Antibacterial agents used in antiviral paints currently on the market are largely classified into ceramic types obtained by melting silver or copper together with glass (silicate) at a high temperature, then cooling and crystallizing, and then pulverizing, and Zeolites such as silver or copper ion-substituted zeolites (i.e., Ag⁺/zeolite or Cu²⁺/zeolite) or cuprous oxide nanoparticle zeolites (Cu₂O NPs/zeolite). However, their manufacturing process is very complicated, such as requiring high-temperature melting or requiring zeolite metal substitution reaction. In addition, after manufacturing, a separate grinding process using an air jet mill or the like is required to have a unit of several microns or less, and the ratio of these powders (i.e., pulverized powder) added to the paint need to be generally 0.3% by weight (3,000 ppm) or more to exert an antibacterial effect. Therefore, the limitation of price/performance has emerged as a problem.

On the other hand, architectural paints and personal care (shampoo, etc.) products prior to requiring antiviral properties have mainly contained zinc pyrithione at a level of 1,000 to 5,000 ppm. However, according to European Chemicals Agency (ECHA) data, the acute oral toxicity of zinc pyrithione to rodents has been reported as LD50 (50% lethal dose) of 269 mg/kg. Therefore, in the case of Korea, there is a concern that it may be classified as a substance with the acute oral toxicity to rodents of LD50 300 mg/kg or less (i.e., toxic substance), which is the standard for designating toxic substances in Article 16 of the Act on Registration and Evaluation of Chemical Substances.

According to literature, the minimum inhibitory concentration of copper ions against Staphylococcus epidermidis is known to be 9 to 90 ppm. This is to demonstrate effective antimicrobial activity at a very low concentration compared to 1,000 to 5,000 ppm, which is the appropriate concentration for zinc pyrithione to exhibit antimicrobial activity. For reference, the acute oral toxicity of copper citrate to rodents is 1,580 mg/kg, which is relatively safe.

The source of copper ions may generally have the form of a simple salt such as cupric chloride (CuCl₂) or copper sulfate (CuSO₄). However, since it is very soluble in water, it cannot be used in paints due to leaching problems. Therefore, it is urgently required to develop an antimicrobial water-dispersive composition for paint and personal care products with antiviral properties that zinc pyrithione does not have, by seeking a copper salt that is insoluble in water and has an excellent antibacterial effect even when used in a small amount.

### DISCLOSURE

### Technical Problem

While searching for a water-insoluble copper salt as described above, in particular, the present applicant came up with complex compounds of organic weak acid and copper. These compounds can be compounds of various combinations, such as Cu-EDTA, Cu-Oxalate, Cu-Aspirinate, and Cu-Citrate. However, the present applicant selected copper citrate (Cu-citrate), which has relatively low toxicity and is simple to prepare. In fact, copper citrate is also taken as a health supplement (copper ion supplement), and is also used as a processing aid during wine production to remove sulfide-based substances that cause unpleasant odors accompanied during the wine manufacturing process such as fermentation.

Accordingly, an object of the present invention is to provide an antimicrobial composition that has excellent water dispersion stability and exhibits a wide antibacterial spectrum against bacteria, fungi and viruses when added to personal care products and paints.

### Technical Solution

In order to achieve the above object, the present invention provides an antimicrobial water-dispersive composition including copper citrate as an active ingredient.

### Advantageous Effects

The antimicrobial water-dispersive composition including copper citrate as an active ingredient according to the present invention has the advantages of having excellent water dispersion stability and exhibiting a wide antibacterial spectrum against bacteria, fungi and viruses when added to personal care products and paints. In addition, the antimicrobial water-dispersive composition including copper citrate as an active ingredient according to the present invention has the advantage of being relatively safe to the human body and securing sufficient antibacterial activity even in a small amount. In addition, the antimicrobial water-dispersive composition including copper citrate as an active ingredient according to the present invention has the advantage of being able to industrially utilize copper citrate, which has been widely used mainly in the Western world.

### DESCRIPTION OF DRAWINGS

FIG. 1(a) is an image after cultivation of S. aureus strain of Control used in the Examples and the Comparative Examples of the present invention and FIG. 1(b) is an image of S. aureus strain killed after contact with a sample according to the present invention.

FIG. 2(a) is an image after cultivation of E. coli strain of Control used in the Examples and the Comparative Examples of the present invention and FIG. 2(b) is an image of E. coli strain killed after contact with a sample according to the present invention.

### BEST MODE

Hereinafter, the present invention will be described in detail.

The terms or words used in this specification and claims should not be construed as being limited to their ordinary or dictionary meanings. Based on the principle that the inventor can appropriately define the concept of the term in order to explain his or her invention in the best way, it should be interpreted as meaning and concept consistent with the technical spirit of the present invention.

An antimicrobial water-dispersive composition according to the present invention is one that contains a large amount of copper ions and can impart antibacterial or antiseptic activity to personal care products (e.g., shampoo, soap and cosmetics) and paints (especially water-based architectural paints). It includes copper citrate as an active ingredient, and may further include any one or more of metal oxide, dispersant, and auxiliary additive, if necessary.

Common architectural paints and personal care products have mainly contained zinc pyrithione at a level of 1,000 to 2,500 ppm. However, while zinc pyrithione has excellent antifungal and antibacterial activity, there is no report on antiviral performance. Therefore, although there is a demand for architectural paints and personal care products having antiviral properties in accordance with the recent global pandemic of corona virus infection (COVID-19), the use of zinc pyrithione can be said to be limited.

Accordingly, considering that the minimum inhibitory concentration of copper ions against Staphylococcus epidermidis is known to be 9 to 90 ppm and these copper ions exhibit effective antimicrobial activity at a very low concentration compared to the appropriate concentration (1,000 to 2,500 ppm) of zinc pyrithione for exhibiting antimicrobial activity, the present applicant focused on complex compounds of organic weak acid and copper such as Cu-EDTA, Cu-Oxalate, Cu-Aspirinate and Cu-Citrate among copper salts insoluble in water. Among them, copper citrate (Cu-citrate), which has relatively low toxicity (acute oral toxicity to rodents: 1,580 mg/kg) and is simple to manufacture, was selected as a key component of the antimicrobial water-dispersive composition. For reference, acute oral toxicity data of the copper complex compounds except for copper citrate have not been reported, the rodent oral toxicity (LD50) data for ‘conjugate acid of copper ion’ was referred to and summarized as shown in Table 1 below.

TABLE 1

| Conjugate acid | LD50(oral, rat) | |
|---|---|---|
| EDTA | 30 | mg/kg |
| Acetylsalicylic acid | 200 | mg/kg |
| Oxalic acid | 375 | mg/kg |
| Citric acid | 11,700 | mg/kg |

Copper citrate, which is included as a key active ingredient in the antimicrobial water-dispersive composition of the present invention, is prepared according to the following Reaction Formula I. At this time, the copper citrate prepared is precipitated in the form of a water insoluble hydrate of the following Formula I, and when it is filtered and dried, copper citrate anhydride represented by the following Formula II below can be obtained. On the other hand, the copper citrate anhydride can be re-converted to the copper citrate hydride by contacting moisture or water in the atmosphere.

3CuSO₄(aq)+2Na₃C₆H₅O₇(aq)→Cu₃(C₆H₅O₇)₂(s)+3Na₂SO₄(aq) [Reaction Formula I]

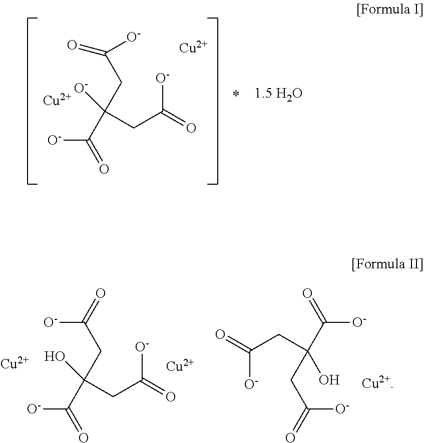

On the other hand, the copper citrate included in the antimicrobial water-dispersive composition of the present invention may be included in the form of a hydrate represented by the above Formula I or an anhydride represented by the above Formula II without limitation (That is, regardless of whether the copper citrate is included in the antimicrobial water-dispersive composition in the form of hydrate or anhydride, the copper citrate always exists in the form of hydrate in the water-dispersive composition.).

However, when it is included in the form of hydrate in an undried state, the weight of copper citrate anhydride added after drying changes according to the moisture content of the copper citrate hydride filter cake. Therefore, it may not be easy to accurately manage the content of copper ions included in the composition product as intended. Accordingly, when considering this aspect, it may be more preferable to include the copper citrate anhydride represented by the above Formula II in the composition.

Here, the copper citrate hydride represented by the above Formula I is (Cu₂C₆H₄O₇)·1.5H₂O in the form of bluish-green copper citrate hemitrihydrate (having a powder form as a precipitate), and the copper citrate anhydride represented by Formula II is Cu₃(C₆H₅O₇)₂ in the form of a sky blue powder.

The particle size of the copper citrate powder according to the present invention may be 0.1 to 100 μm, preferably 1 to 20 μm. If the particle size of the copper citrate powder is less than 0.1 μm, a separate grinding process may be required or a disadvantageous problem may occur in terms of worker safety due to fine dust during the process. If it exceeds 100 μm, it is out of the particle size management standard of raw materials applied to paints or personal care products (300 Mesh(=50 μm) 99.8% or more passes), or the release of copper ions is reduced by contact with moisture in the air due to the decrease in the surface area of the particles, which can lead to a decrease in antibacterial activity.

In addition, the average particle diameter (D50) of the copper citrate powder according to the present invention may be 0.5 to 10 μm, preferably 1 to 5 μm. If the average particle diameter (D50) of the copper citrate powder is less than 0.5 μm, problems may occur in the process of filtering and washing of the copper citrate powder prepared by the above Reaction Formula I, and if it exceeds 10 μm, layer stability of the water-dispersive composition, including precipitation, may deteriorate, resulting in problems with product storage stability.

The copper citrate may be included in an amount of 0.1 to 50% by weight, preferably 15 to 45% by weight, based on the total weight of the antimicrobial water-dispersive composition of the present invention. If the copper citrate is included in an amount of less than 0.1% by weight based on the total weight of the antimicrobial water-dispersive composition, the low concentration of copper citrate may cause problems with excessive logistics costs and insufficient antibacterial activity, and if it is included in an amount exceeding 50% by weight, an increase in viscosity and poor flowability of the water-dispersive composition due to the excessive solid content in the water-dispersive composition product may cause problems with ease of use.

As such copper citrate is included in the composition, the antimicrobial water-dispersive composition of the present invention may contain a large amount of copper ions. More specifically, the content of copper ions contained in the antimicrobial water-dispersive composition may be 0.3 to 170 mg/mL, preferably 50 to 100 mg/mL. If the content of copper ions in the antimicrobial water-dispersive composition is less than 0.1 mg/mL, the antibacterial ability of the composition may be lowered, resulting in a problem of not exhibiting a wide antibacterial spectrum against bacteria, fungi, and viruses, and if it exceeds 170 mg/mL, problems may arise in the ease of use of the water-dispersive composition.

On the other hand, the antimicrobial water-dispersive composition including copper citrate as an active ingredient according to the present invention essentially includes pure water (DI water) in addition to the copper citrate, and may further include any one or more of metal oxide, dispersant, and auxiliary additive, if necessary. All of these metal oxides, dispersants, and auxiliary additives can play a role in providing synergistic antibacterial effects with copper citrate.

In addition, the metal oxide is generally used in the field of personal care and paint, and in order to minimize the compositional heterogeneity in the formulation of personal care products and paints that have been used so far and to promote the formulation stability of the finished product, the metal oxide can also be included in the antimicrobial water-dispersive composition of the present invention.

Examples of such metal oxides include zinc oxide (ZnO), titanium dioxide (TiO₂), silica (SiO₂), alumina (Al₂O₃), and mixtures thereof. If the metal oxide is included in the antimicrobial water-dispersive composition of the present invention, it may be included in an amount of 20% by weight or less, preferably 10 to 15% by weight, based on the total weight of the antimicrobial water-dispersive composition.

The dispersant is preferably a surfactant, and as such a surfactant, those commonly used in the art may be used without limitation. Specifically, examples thereof include Sodium lauryl sulfate, Polyoxyethylene sorbitan monooleate (Product name: Tween 80), Polyethylene glycol tert-octylphenyl ether (Product name: Triton X-100), Sorbitan monopalmitate (Product name: Span 40), and mixtures thereof. If the dispersant is included in the antimicrobial water-dispersive composition of the present invention, it may be included in an amount of 0.1 to 1% by weight, preferably 0.5 to 0.8% by weight, based on the total weight of the antimicrobial water-dispersive composition.

The auxiliary additive is preferably a thickener, and as such a thickener, those commonly used in the art may be used without limitation. Specifically, examples thereof include Methyl cellulose, Hydroxypropyl methyl cellulose, Carboxy methyl cellulose, Xanthan Gum, Polyacrylic acid (Product name: Carbopol) and mixtures thereof. If the auxiliary additive is included in the antimicrobial water-dispersive composition of the present invention, it may be included in an amount of 0.1 to 3% by weight, preferably 0.7 to 1.5% by weight, based on the total weight of the antimicrobial water-dispersive composition.

On the other hand, another object of the present invention is to industrially utilize the copper citrate, which has been widely used mainly by the Western civilians. The copper citrate production method in the private sector is generally based on an electrochemical method. Specifically, it is a method of obtaining a low-concentration copper citrate aqueous solution in a colloidal state by inserting a copper electrode into water in which citric acid is dissolved and passing an electric current. The concentration of copper citrate in the colloidal state obtained at this time varies depending on the applied voltage, amount of current and time, but generally a small amount of copper citrate of around 10 ppm is obtained in 6 hours. Therefore, copper citrate cannot be industrially utilized in this way.

Accordingly, the present invention provides a method for manufacturing an antimicrobial water-dispersive composition including copper citrate as an active ingredient, which chemically synthesizes copper citrate in large quantities to improve convenience and industrial utilization.

The method for manufacturing the antimicrobial water-dispersive composition including copper citrate as an active ingredient according to the present invention includes: a) a step of mass-synthesizing copper citrate by reacting copper sulfate and sodium citrate according to the following Reaction Formula I: and b) a step of adding the synthesized copper citrate to pure water (DI water).

If necessary, any one or more of metal oxide, dispersant, and auxiliary additive may be further added together with the copper citrate in the step b). In addition, if necessary, a grinding process using a bead mill or the like may be performed after the step b).

3CuSO₄(aq)+2Na₃C₆H₅O₇(aq)→Cu₃(C₆H₅O₇)₂(s)+3Na₂SO₄(aq) [Reaction Formula I]

On the other hand, the copper citrate synthesized or manufactured in Reaction Formula I above is precipitated in the form of copper citrate hydride. In order to secure the quantification of the copper ion concentration, it may be desirable to convert the copper citrate hydride into a form of copper citrate anhydride by filtering the copper citrate hydride and drying it completely.

The antimicrobial water-dispersive composition including copper citrate as an active ingredient of the present invention described above can be widely used in fields requiring antibacterial properties such as personal care and paint. Therefore, it can be included and used in products requiring antibacterial properties such as personal care products and paints (particularly, water-based paints), and at this time, it can be commercialized using a bead mill or the like.

Hereinafter, preferred embodiments are presented to help understanding of the present invention. However, the following examples are merely illustrative of the present invention, and it is obvious to those skilled in the art that various changes and modifications are possible within the scope and spirit of the present invention, and it is natural that these changes and modifications fall within the scope of the appended claims.

### [Example 1] Preparation of Antimicrobial Water-Dispersive Composition Containing Copper Citrate

First, 413.7 g of pure water (DI water) was put into a 600 ml beaker. 75.0 g of copper citrate anhydride powder prepared by reacting 374.5 g (1.5 mol) of copper sulfate pentahydrate and 294.1 g (1.0 mol) of sodium citrate dihydrate, 7.75 g of Hydroxypropyl methyl cellulose (thickener) and 3.55 g of Sodium lauryl sulfate (surfactant) were added thereto, and then stirred and dispersed at a temperature of 30° C. for 3 hours to prepare 500 g of an antimicrobial water-dispersive composition.

### [Example 2] Preparation of Antimicrobial Water-Dispersive Composition Containing Copper Citrate

The procedure of Example 1 was repeated except that, instead of reducing the content of pure water (DI water) by 50.0 g from 413.7 g to 363.7 g, the amount of zinc oxide equal to the amount of reduced pure water was additionally added to prepare 500 g of an antimicrobial water-dispersive composition.

### [Example 3] Preparation of Antimicrobial Water-Dispersive Composition Containing Copper Citrate

The procedure of Example 2 was repeated except that, instead of increasing the content of pure water (DI water) by 67.5 g from 363.7 g to 431.2 g, the content of copper citrate anhydride powder was reduced by the amount of increased pure water to prepare 500 g of an antimicrobial water-dispersive composition.

### [Comparative Example 1] Preparation of Conventional Structural Adhesive Composition

Except for using Cu²⁺/zeolite instead of copper citrate, a water-dispersive composition was prepared by mixing in the same composition as in Example 1 above. On the other hand, the manufacturing method of Cu²⁺/zeolite is a known technology, and the present applicant self-manufactured and used it.

Each composition of the compositions prepared in Examples 1 to 3 and Comparative Example 1 above is shown in Table 2 below.

TABLE 2

| | Copper citrate | Cu²⁺/ | | | Zinc | Pure water | |
|---|---|---|---|---|---|---|---|
| | (Anhydride) | zeolite | Thickener | Surfactant | oxide | (DI water) | Total |
| Example 1 | 75.0 | — | 7.75 | 3.55 | — | 413.7 | 500 |
| Example 2 | 75.0 | — | 7.75 | 3.55 | 50.0 | 363.7 | 500 |
| Example 3 | 7.5 | — | 7.75 | 3.55 | 50.0 | 431.2 | 500 |
| Comparative | — | 75.0 | 7.75 | 3.55 | — | 413.7 | 500 |
| Example 1 | | | | | | | |

※ Unit: gram(g)

### [Test Example 1] Measurement of Concentration of Copper Ion in Composition

The concentration (%) of copper ion (Cu²⁺) included in the compositions prepared in Examples 1 to 3 and Comparative Example 1 was measured, and the results are shown in Table 3 below.

TABLE 3

| | Concentration of Copper ion(Cu²⁺)(Unit: %) |
|---|---|
| Example 1 | 5.0 |
| Example 2 | 5.0 |
| Example 3 | 0.5 |
| Comparative Example 1 | 1.1 |

### [Test Example 2] Evaluation of Antimicrobial Activity of Composition

Antimicrobial activity against two bacteria (S. aureus and E. coli) of each of the compositions prepared in Example 1 to 3 and Comparative Example 1 above was evaluated. The evaluation method for bacteria was performed according to ASTM E 2149. Bacteria pre-cultured for 18 hours was inoculated in 50 mL phosphate buffer solution to make the concentration of bacteria of 1.5 to 3.0×10⁵ CFU/mL. After adding 0.1 g (=0.2%) of each sample and shaking for 1 hour, the CFU (Colony Forming Unit) of the buffer was measured and the bacteria reduction rate was measured according to Equation 1 below, and the results are shown in Table 4 below. Meanwhile, as shown in Table 3 above, the copper ion concentration in the composition prepared in Comparative Example 1 is 1.1%, which is lower than the copper ion concentration (5.0%) in the composition prepared in Example 1. Therefore, in order to meet the same copper ion concentration conditions, the amount of the sample corresponding to Comparative Example 1 was increased to 0.5 g (=1.0%).

$$Bacteria reduction rate ( \% ) = [ ( Bacterial count after incubation of Control ) - ( Bacterial count after incubation of Sample ) ] / ( Bacterial count after incubation of Control ) \times 100 \qquad [ Equation I ]$$

TABLE 4

| | | Bacteria reduction rate | | | |
|---|---|---|---|---|---|
| Test | Subject | | | | Comparative |
| Method | Strain | Example 1 | Example 2 | Example 3 | Example 1 |
| ASTM E | S. aureus | 98.6% | 99.1% | 98.1% | 96.3% |
| 2149 | (ATCC | | | | |
| | 6538) | | | | |
| | E. coli | 99.9% | 99.9% | 98.6% | 97.2% |
| | (ATCC | | | | |
| | 8739) | | | | |

As a result of the evaluation, the antimicrobial water-dispersive composition of Examples 1 to 3 including copper citrate showed a bacterial reduction rate of 98.1 to 99.9% within 1 hour of contact with bacteria. On the other hand, Comparative Example 1 using Cu²⁺/zeolite, a common antibacterial agent, showed a bacterial reduction rate of 96.3 to 97.2%. Therefore, it was confirmed that Comparative Example 1 had lower antibacterial performance than Examples 1 to 3. In addition, through comparison and contrast between Example 1 including only copper citrate, thickener, and surfactant and Example 2 including zinc oxide in addition to the composition of Example 1, it was confirmed that the antibacterial performance against S. aureus was slightly improved when zinc oxide was added to the composition.

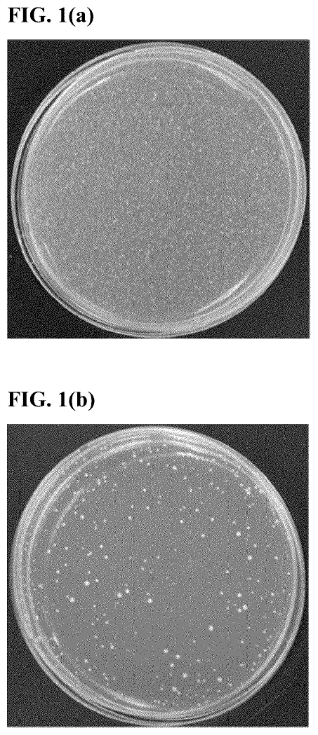

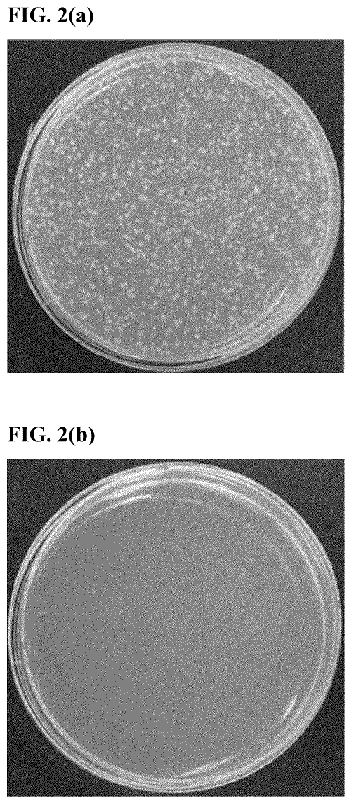

On the other hand, FIG. 1(a) is an image after cultivation of S. aureus strain of Control used in the Examples and the Comparative Examples of the present invention and FIG. 1(b) an image of S. aureus strain killed after contact with the sample of Example 1. FIG. 2(a) is an image after cultivation of E. coli strain of Control used in the Examples and the Comparative Examples of the present invention and FIG. 2(b) an image of E. coli strain killed after contact with the sample of Example 1.

However, in the case of Cu²⁺/zeolite included in the composition of Comparative Example 1, the concentration of copper ions in the water-dispersive composition is only 1.1%. Therefore, despite setting the same copper ion concentration as in Examples 1 and 2 by increasing the amount of the sample used for the antibacterial activity test by about 5 times, the bacterial reduction rate was 96.3 to 97.2%, which was inferior to the bacterial reduction rate of the copper citrate.

Meanwhile, according to literature, the rodent acute oral toxicity (LD 50) of Cu²⁺/zeolite containing copper ions at a concentration of 2.5% by weight is known to be 18,000 mg/kg. Considering that pure copper citrate (copper ion concentration: 33.5%) has a rodent acute oral toxicity (LD 50) of 1,580 mg/kg, it can be inferred that copper citrate is less harmful to the human body than Cu²⁺/zeolite.

1. An antimicrobial water-dispersive composition comprising copper citrate as an active ingredient.

2. The antimicrobial water-dispersive composition according to claim 1, wherein the copper citrate is copper citrate hydride represented by the following Formula I or copper citrate anhydride represented by the following Formula II:

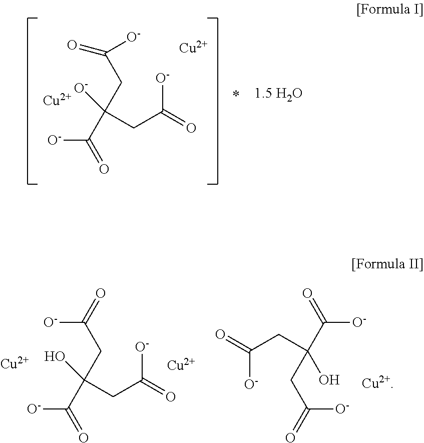

3. The antimicrobial water-dispersive composition according to claim 2, wherein the copper citrate hydride and the copper citrate anhydride have a powder form.

4. The antimicrobial water-dispersive composition according to claim 3, wherein a particle size of the copper citrate powder is 0.1 to 100 μm, and an average particle diameter (D50) of the copper citrate powder is 0.5 to 10 μm.

5. The antimicrobial water-dispersive composition according to claim 1, wherein the copper citrate is included in an amount of 0.1 to 50% by weight based on the total weight of the composition.

6. The antimicrobial water-dispersive composition according to claim 1, wherein a content of copper ions contained in the antimicrobial water-dispersive composition is 0.3 to 170 mg/mL.

7. The antimicrobial water-dispersive composition according to claim 1, wherein the antimicrobial water-dispersive composition is composed of copper citrate and water.

8. The antimicrobial water-dispersive composition according to claim 1, wherein the antimicrobial water-dispersive composition further comprises any one or more of metal oxides, dispersants and auxiliary additives.

9. The antimicrobial water-dispersive composition according to claim 8, wherein the metal oxide is selected from the group consisting of zinc oxide (ZnO), titanium oxide (TiO₂), silica (SiO₂), alumina (Al₂O₃) and mixtures thereof.

10. The antimicrobial water-dispersive composition according to claim 8, wherein the dispersant is a surfactant selected from the group consisting of a sodium lauryl sulfate, a polyoxyethylene sorbitan monooleate, a polyethylene glycol tert-octylphenyl ether, a sorbitan monopalmitate, and mixtures thereof.

11. The antimicrobial water-dispersive composition according to claim 8, wherein the auxiliary additive is a thickener selected from the group consisting of a methyl cellulose, a hydroxypropyl methyl cellulose, a carboxy methyl cellulose, a xanthan gum, a polyacrylic, acid and mixtures thereof.

12. The antimicrobial water-dispersive composition according to claim 1, wherein the antimicrobial water-dispersive composition is intended for use in personal care products or paints.

13. A personal care product comprising the antimicrobial water-dispersive composition of claim 1.

14. A paint composition comprising the antimicrobial water-dispersive composition of claim 1.
